(19) United States
(12) Patent Application Publication
(10) Pub. No.: US 2005/0065579 A1
(43) Pub. Date: Mar. 24, 2005

(54) Portable infrared device

(76) Inventors: George C.C. Chen, Taipei (TW); George Chiang, Cheshan Township (TW)

Correspondence Address: DENNISON, SCHULTZ, DOUGHERTY & MACDONALD, Suite 105, 1727 King Street, Alexandria, VA, 22314, US

(21) Appl. No.: 10/756,265

(22) Filed: Jan. 14, 2004

(30) Foreign Application Priority Data
Aug. 18, 2003 (TW) 092214882

(51) Int. Cl. A61N 1/00

(52) U.S. Cl. 607/88

(57) ABSTRACT

Disclosed is a portable IR device for the treatment of skin. The device comprises a handle, a light-emitting assembly including one or more replaceable light-emitting elements formed of either the same LED for emitting IR rays of a predetermined wavelength or different LEDs for emitting IR rays of different wavelengths, and a controller assembly coupled to the light-emitting assembly for controlling irradiation time, wavelength of IR rays, and light-emitting mode of each light-emitting element. Holding the handle and activating the device will cause the light-emitting elements to emit IR rays for treating the skin in close proximity therewith.

### BACKGROUND OF THE INVENTION

1. Field of the Invention

The present invention relates to an irradiating device and more particularly to a portable device for emitting infrared rays and shining the same upon the skin so as to enhance metabolism of the skin.

2. Description of Related Art

Exposing skin to light for treatment is always a significant concern. A prior pulsed light device is adapted to emit rays of high energy and different wavelengths. But rather than all rays of different wavelengths being emitted at one time only a selected ray is emitted for a specific purpose when the device is operating. In a medical application, a doctor has to choose rays having an appropriate wavelength and decide the duration of irradiation prior to activating the device for treatment. Otherwise, it may damage tissue in the irradiated area of the skin. Hence, for providing a good treatment, a plurality of parameters (e.g., wavelength, strength of radiant energy, number of pulses, irradiation time, stop time, etc.) have to set prior to operating the device. Such parameter setting is a difficult task. Typically, only an experienced doctor is qualified. Moreover, the device involves using filters to obtain rays of different wavelengths which in turn, are processed to output pulsed light. Conventionally, the pulsed light device comprises a reflector, filters, a flashlight, and related electronic elements for controlling the parameters (e.g., wavelength, strength of radiant energy, number of pulses, irradiation time, stop time, etc.) of the pulsed light. Inevitably, it makes the device bulky and thus prohibits the portability thereof from being made possible. Hence, a need for improvement exists.

### SUMMARY OF THE INVENTION

It is an object of the present invention to provide a portable infrared device for the treatment of skin. A user can operate the device without setting parameters in advance. Further, the device will not hurt or burn the skin being treated. Furthermore, a user can conveniently operate the device at any place any time. Moreover, the device can enhance metabolism of the skin so as to be applicable to medical care or beauty purpose.

To achieve the above and other objects, the present invention provides a portable infrared device for the treatment of skin, comprising a housing including a handle, a first housing, and a lens for spreading IR rays passing therethrough; a light-emitting assembly in the housing, the light-emitting assembly including one or more light-emitting elements formed of either the same LED for emitting IR rays of a predetermined wavelength or different LEDs for emitting IR rays of different wavelengths, the light-emitting elements being arranged as a ring; and a controller assembly in the housing, the controller assembly being coupled to the light-emitting assembly for controlling an irradiation time, a wavelength of IR rays, and a light-emitting mode of each of the light-emitting elements, whereby holding the handle and activating the device will cause the light-emitting elements to emit IR rays for treating the skin in close proximity therewith.

In one aspect of the present invention the controller assembly comprises a controller for issuing a plurality of trigger signals; a timer unit coupled to the controller and the light-emitting assembly respectively so that the controller is operative to enable the light-emitting assembly by conducting the timer unit by means of the trigger signal; and a power adjustment unit coupled to the timer unit for adjusting output power of the light-emitting assembly.

In another aspect of the present invention the trigger signals are DC signals so that the light-emitting assembly are adapted to continuously emit IR rays of a predetermined wavelength in any period of time.

In still another aspect of the present invention the trigger signals are AC signals so that the light-emitting assembly are adapted to intermittently emit IR rays of a predetermined wavelength in any period of time.

In a further aspect of the present invention the controller assembly further comprises a buzzer adapted to make a buzzing sound when the irradiation time expires. Next, the controller disables the light-emitting assembly for timing.

The above and other objects, features and advantages of the present invention will become apparent from the following detailed description taken with the accompanying drawings.

### BRIEF DESCRIPTION OF THE DRAWINGS

FIG. 1A is a front view of a portable infrared device according to the invention;

FIG. 1B is a rear view of the infrared device;

FIG. 1C is a side view of the infrared device;

FIG. 2 is a plan view of light-emitting assembly according to a second preferred embodiment of the invention;

FIG. 3 is a plan view of light-emitting assembly according to a third preferred embodiment of the invention;

FIG. 4 is a plan view of light-emitting assembly according to a fourth preferred embodiment of the invention;

FIG. 5 is a plan view of light-emitting assembly according to a fifth preferred embodiment of the invention; and

FIG. 6 is an electrical block diagram of the invention.

### DETAILED DESCRIPTION OF THE PREFERRED EMBODIMENTS

The skin treatment referring in the descriptions of the present invention comprises the skin healing, skin care, skin disease and dermotherapy . . . etc.

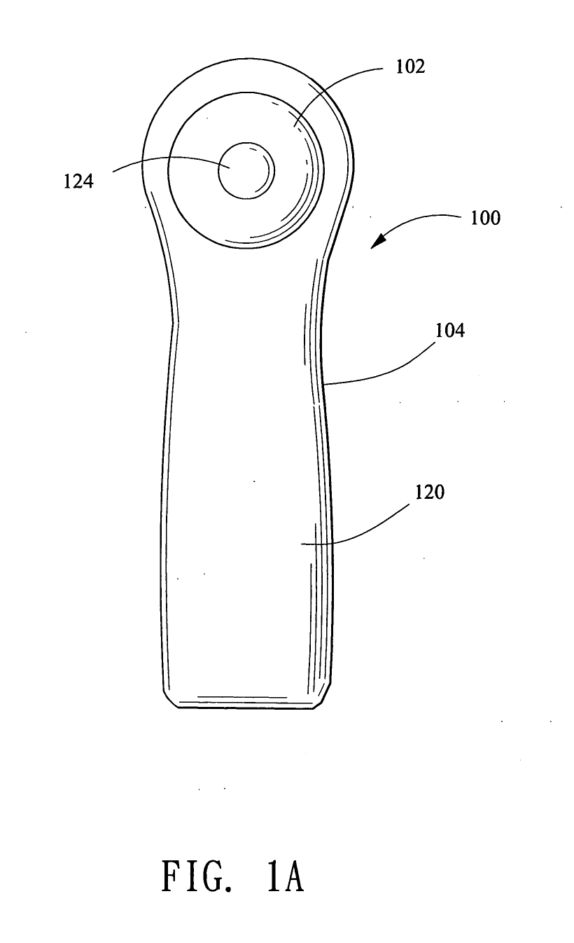

Referring to FIGS. 1A, 1B, and 1C, a portable infrared (IR) device 100 constructed in accordance with the invention is shown. The device 100 comprises a lens 102, a first housing 104, a handle 120 (see FIG. 1A), a switch 106, a second housing 108 (see FIG. 1B), a circuit board 110, a light-emitting assembly 112 according to a first preferred embodiment of the invention, signal lines 114 and 116, and a power cord 118 for supplying external power to the circuit board 110 (see FIG. 1C). The lens 102 is provided on an upper of the first housing 104. The first housing 104 and the second housing 108 are combined together to form a housing 122. The circuit board 110 is electrically coupled to the switch 106 and the light-emitting assembly 112 via the signal lines 116 and 114 respectively. Both the circuit board 110 and the light-emitting assembly 112 are provided in the housing 122. The light-emitting assembly 112 comprises a replaceable light-emitting element 124.

The operation of the device 100 will be described in detail below. A pressing of the switch 106 will enable the circuit board 110. Next, the light-emitting assembly 112 is activated to emit IR rays of a predetermined wavelength. The emitted IR rays can be shone upon the skin for treatment or some other purpose. By operating the device 100, it is possible of further eliminating black spots or healing a blood vessel disease by cooperating with another irradiating device. Note that the device 100 can contact the skin directly without involving medium.

For example, if a user wants to eliminate black spots the user may hold the handle 120 to press the switch 106 with the lens 102 being in close proximity with the black spots. Next, the light-emitting assembly 112 is activated to emit IR rays of a predetermined wavelength and shine the same upon the black spots. The device 100 will stop the irradiation automatically once a predetermined period time has reached. Otherwise, it may hurt or burn the skin adjacent the black spots. A light-emitting mode of the light-emitting element 124 may be set as one of two modes. The first mode involves continuously emitting IR rays of a predetermined wavelength in any period of time. The second mode involves intermittently emitting IR rays of a predetermined wavelength in any period of time. It is important to note that only a simple pressing of the switch 106 is required in operation since wavelength, irradiation time, and light-emitting mode of the light-emitting element 124 are set in advance by the manufacturer.

In the embodiment, for obtaining an optimum effect of the device 100, the irradiation time is set as about 5 to 15 minutes, the adjustable power is set about 10 mW to 50 mW subject to the selected light-emitting material, the output frequency of IR rays is set about 130 Hz to 200 Hz, and the wavelength of IR rays is set about 400 nm to 1,500 nm subject to the selected LED (light-emitting diode) material. The device 100 will not hurt or burn the skin in operation. Also, there is no need to apply balm medium or the like on the skin prior to or during operation. These are characteristics of the invention.

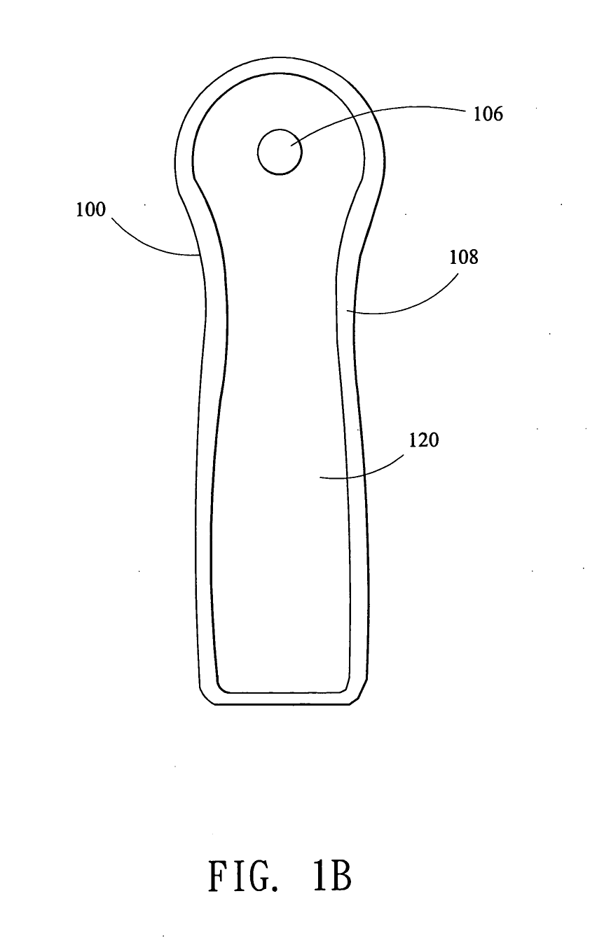

Referring to FIG. 2, a light-emitting assembly 212 according to a second preferred embodiment of the invention is shown. The light-emitting assembly 212 of the second preferred embodiment substantially operates the same function as the light-emitting assembly 112 of the first preferred embodiment. Thus a detailed description thereof is omitted herein for the sake of brevity. The differences between the light-emitting assemblies of the first and the second preferred embodiments, i.e., the characteristics of the light-emitting assembly 212 are detailed below. The light-emitting assembly 212 comprises three light-emitting elements 202, 204, and 206 arranged in a shape (e.g., triangle) based on a predetermined irradiation area and any other factors. The light-emitting elements 202, 204, and 206 are adapted to emit IR rays of the same wavelength if they are made of the same LED material. Alternatively, the light-emitting elements 202, 204, and 206 are adapted to emit three types of IR rays each having a different wavelength if they are made of three different LED materials. In other words, it is possible of changing the wavelength of IR rays emitted by the light-emitting assembly 212 in the later case.

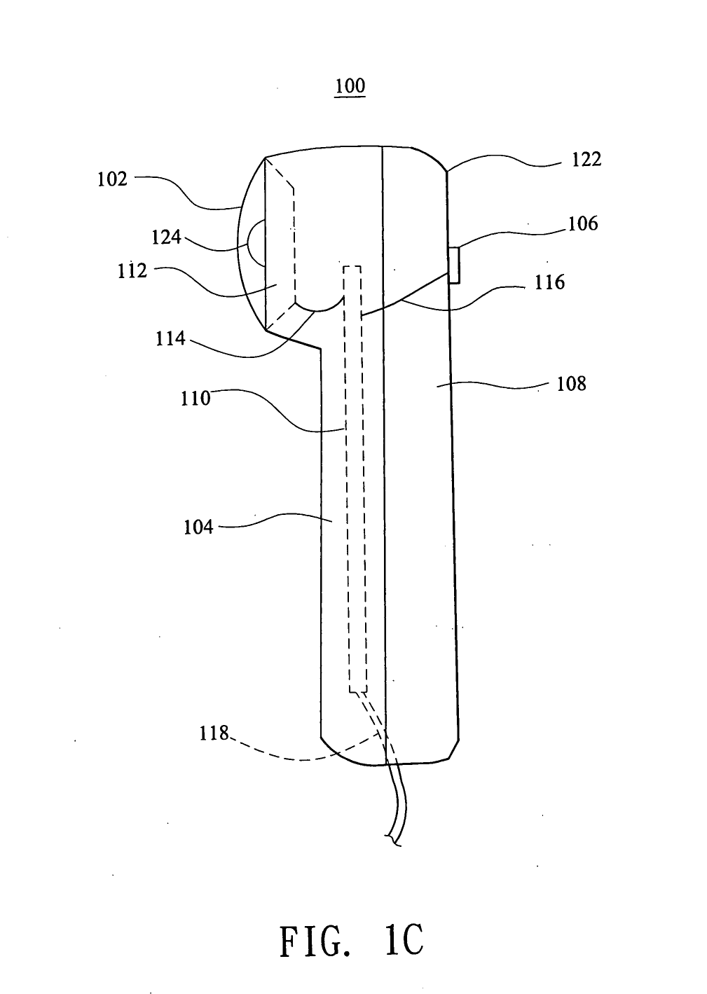

Referring to FIG. 3, a light-emitting assembly 312 according to a third preferred embodiment of the invention is shown. The light-emitting assembly 312 of the third preferred embodiment substantially operates the same function as the light-emitting assembly 212 of the second preferred embodiment. Thus a detailed description thereof is omitted herein for the sake of brevity. The differences between the light-emitting assemblies of the second and the third preferred embodiments, i.e., the characteristics of the light-emitting assembly 312 are detailed below. The light-emitting assembly 312 comprises a light-emitting element labeled “1” made of a first LED material adapted to emit IR rays of a first predetermined wavelength, and six light-emitting elements labeled “2” equally spaced around the light-emitting element labeled “1”, the light-emitting elements labeled “2” being shaped as a circle and being made of a different second LED material adapted to emit IR rays of a second predetermined wavelength. In other words, the light-emitting assembly 312 is able to emit two types of IR rays each having a different wavelength.

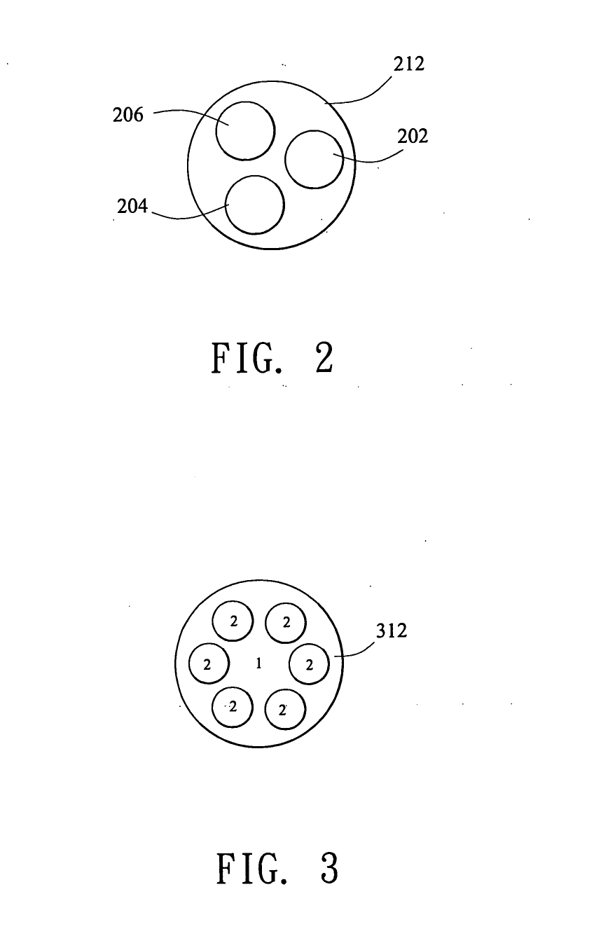

Referring to FIG. 4, a light-emitting assembly 412 according to a fourth preferred embodiment of the invention is shown. The light-emitting assembly 412 of the fourth preferred embodiment substantially operates the same function as the light-emitting assembly 312 of the third preferred embodiment. Thus a detailed description thereof is omitted herein for the sake of brevity. The differences between the light-emitting assemblies of the third and the fourth preferred embodiments, i.e., the characteristics of the light-emitting assembly 412 are detailed below. The light-emitting assembly 412 comprises eight light-emitting elements labeled “2” rather than six light-emitting elements. The light-emitting elements labeled “2” are equally spaced around the light-emitting element labeled “1”. The provision of eight light-emitting elements aims at increasing radiant energy.

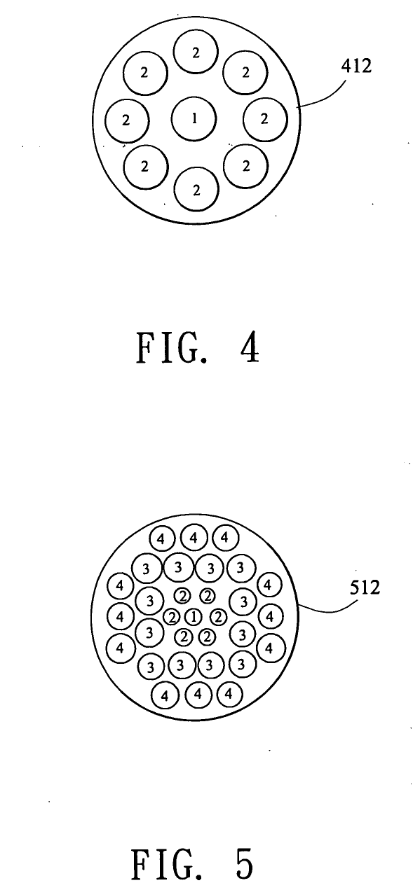

Referring to FIG. 5, a light-emitting assembly 412 according to a fifth preferred embodiment of the invention is shown. The light-emitting assembly 512 of the fifth preferred embodiment substantially operates the same function as the light-emitting assembly 312 of the third preferred embodiment. Thus a detailed description thereof is omitted herein for the sake of brevity. The differences between the light-emitting assemblies of the third and the fifth preferred embodiments, i.e., the characteristics of the light-emitting assembly 512 are detailed below. In addition to the light-emitting elements labeled “1” and “2”, the light-emitting assembly 512 further comprises 12 light-emitting elements labeled “3” shaped as a square around the light-emitting elements labeled “2”, the light-emitting elements labeled “3” being made of a third LED material adapted to emit IR rays of a third predetermined wavelength, and 12 light-emitting elements labeled “4” wherein every three light-emitting elements are proximate one side of the square formed by the light-emitting elements labeled “3”, the light-emitting elements labeled “4” being made of a fourth LED material adapted to emit IR rays of a fourth predetermined wavelength. In other words, the light-emitting assembly 512 is able to emit four types of IR rays each having a different wavelength.

In view of the above, it is seen that the light-emitting elements can be arranged in one of different shapes and the LED materials for emitting different IR rays can be selected depending on applications. Thus, the number of the light-emitting elements, the shapes of the light-emitting elements, and the LED materials described are illustrative only. It is appreciated by those skilled in the art that the above preferred embodiments may be modified without departing from the scope and spirit of the invention.

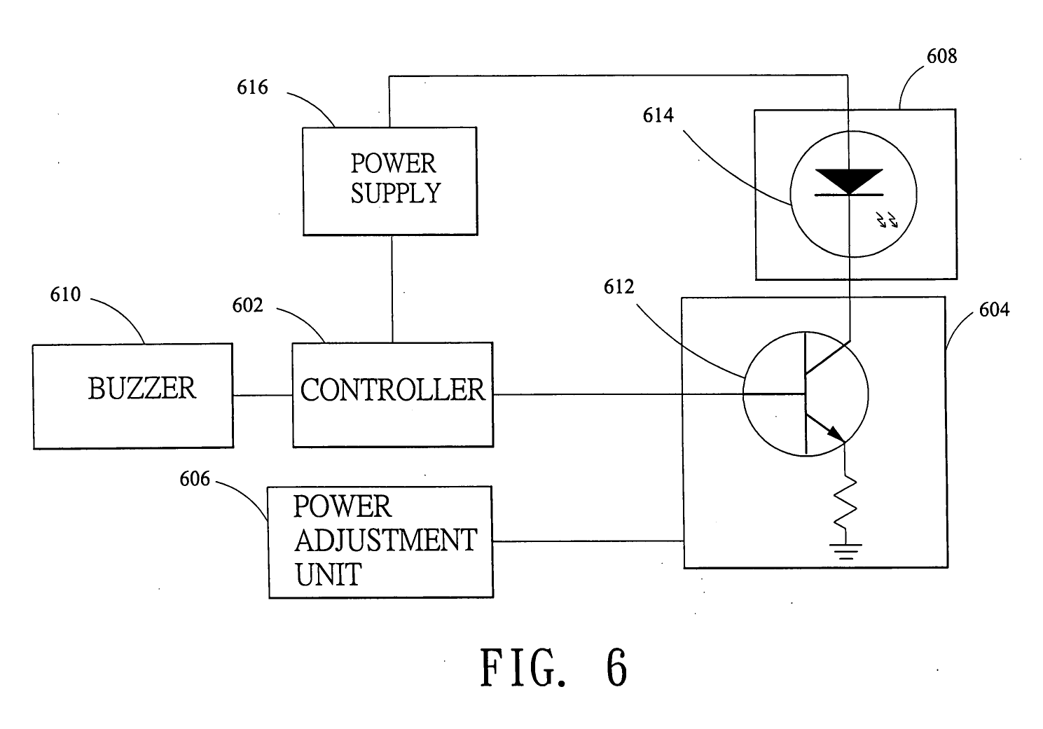

Referring to FIG. 6, the circuit board 110 comprises a controller 602, a timer unit 604, a power adjustment unit 606, a light-emitting assembly 608, a buzzer 610, and a power supply 616. Each component will be described in detail below. The timer unit 604 is coupled to the controller 602, the power adjustment unit 606, and the light-emitting assembly 608 respectively. The controller 602 is coupled to the buzzer 610 and the power supply 616 respectively. The timer unit 604 is implemented as a transistor 612 and the light-emitting assembly 608 is implemented as a LED 614 respectively. The controller 602 is implemented as a microprocessor formed of nonvolatile memory. The controller 602 is adapted to issue a trigger signal to conduct the LED 614 for emitting IR rays via the power supply 616. The trigger signal can be either a direct current (DC) signal so that the timer unit 604 is adapted to continuously emit IR rays of a predetermined wavelength or an alternating current (AC) signal so that the timer unit 604 is adapted to intermittently emit IR rays of a predetermined wavelength in any period of time. Also, the controller 602 is programmed as one having a GPIO (general purpose input/output) for issuing another signal of high voltage level or another AC signal to the base of the transistor 612 for conduction in any period of time. As a result, the irradiation time and the light-emitting mode of the device 100 can be controlled. The power adjustment unit 606 is adapted to adjust the output power of the LED 614. The buzzer 610, as controlled by the controller 602, will make a buzzing sound when the predetermined irradiation time expires. Next, the controller 602 disables the light-emitting assembly 608. It is possible of causing the light-emitting assembly to emit IR rays of many different wavelengths by increasing the number of trigger signals and the number of LEDs. In other words, it is possible of causing the light-emitting assembly to emit IR rays of many different wavelengths by configuring the different LEDs and the trigger signals.

In addition to the above advantages, the invention also has the advantages of being safe, convenient in use, and significant reduction in the manufacturing cost. This is beneficial to consumers.

While the invention herein disclosed has been described by means of specific embodiments, numerous modifications and variations could be made thereto by those skilled in the art without departing from the scope and spirit of the invention set forth in the claims.

1. A portable infrared (IR) device for the treatment of skin, comprising:
a housing including a handle on a lower portion of the housing for hand holding, and a lens on an upper portion of the housing for spreading an IR ray passing therethrough;
a light-emitting assembly in the housing, the light-emitting assembly including one or more light-emitting elements formed of either the same light-emitting diode (LED) for emitting IR rays of a predetermined wavelength or different LEDs for emitting IR rays of different wavelengths; and
a controller assembly in the housing, the controller assembly being coupled to the light-emitting assembly for controlling an irradiation time, a wavelength of IR rays, and a light-emitting mode of each of the light-emitting elements,
whereby holding the handle and activating the device will cause the light-emitting elements to emit IR rays for treating the skin in close proximity therewith.

2. The device of claim 1, wherein the controller assembly comprises:
a controller for issuing a plurality of trigger signals;
a timer unit coupled to the controller and the light-emitting assembly respectively so that the controller is operative to enable the light-emitting assembly by conducting the timer unit by means of the trigger signal; and
a power adjustment unit coupled to the timer unit for adjusting output power of the light-emitting assembly.

3. The device of claim 1, wherein the trigger signals are direct current (DC) signals.

4. The device of claim 1, wherein the trigger signals are alternating current (AC) signals.

5. The device of claim 1, wherein the light-emitting mode involves causing the light-emitting assembly to continuously emit IR rays of a predetermined wavelength in any period of time.

6. The device of claim 1, wherein the light-emitting mode involves causing the light-emitting assembly to intermittently emit IR rays of a predetermined wavelength in any period of time.

7. The device of claim 1, further comprising a switch for turning on/off power.

8. The device of claim 1, wherein the light-emitting elements are arranged as a ring.

9. The device of claim 1, wherein the wavelength of IR rays of each of the light-emitting elements is adapted to change by replacing it with another light-emitting element capable of emitting IR rays of a different wavelength.

10. The device of claim 1, wherein the controller assembly further comprises a buzzer for making a buzzing sound when the irradiation time expires.

11. The device of claim 1, wherein power of the device is adjustable from the range of about 10 mW to about 50 mW.

12. The device of claim 1, wherein output frequency and the wavelength of IR ray of the device are adjustable from the range of about 130 Hz to about 200 Hz and from the range of about 400 nm to 1,500 nm, respectively.

13. A portable IR device for the treatment of skin, comprising:
a housing including a handle, a first housing, a second housing, and a lens;
a light-emitting assembly in the housing, the light-emitting assembly including one or more light-emitting elements for emitting IR rays of a predetermined wavelength wherein the wavelength of IR rays of each of the light-emitting elements is adapted to change by replacing it with another light-emitting element capable of emitting IR rays of a different wavelength; and
a controller assembly in the housing, the controller assembly including:
a controller for issuing a plurality of trigger signals;
a timer unit coupled to the controller and the light-emitting assembly respectively so that the controller is operative to enable the light-emitting assembly by conducting the timer unit by means of the trigger signal, and the light-emitting assembly is operative to either continuously or intermittently emit IR rays of a predetermined wavelength in response; and
a power adjustment unit coupled to the timer unit for adjusting output power of the light-emitting assembly,
whereby holding the handle and activating the device will cause the light-emitting elements to emit IR rays for treating the skin in close proximity therewith.

14. The device of claim 13, wherein the light-emitting elements are formed of LEDs arranged as a ring.

15. The device of claim 13, wherein the trigger signals are DC signals so that the light-emitting assembly are adapted to continuously emit IR rays of a predetermined wavelength in any period of time.

16. The device of claim 13, wherein the trigger signals are AC signals so that the light-emitting assembly are adapted to intermittently emit IR rays of a predetermined wavelength in any period of time.

17. The device of claim 13, further comprising a switch for turning on/off power.

18. The device of claim 13, further comprising a power cord for supplying external power to the device.

19. The device of claim 13, wherein power of the device is adjustable from the range of about 10 mW to about 50 mW.

20. The device of claim 13, wherein output frequency and the wavelength of IR ray of the device are adjustable from the range of about 130 Hz to about 200 Hz and from the range of about 400 nm to 1,500 nm, respectively.
